(19) United States
(12) Patent Application Publication
(10) Pub. No.: US 2025/0222243 A1
(43) Pub. Date: Jul. 10, 2025

(54) INSTANT MEMBRANE

(71) Applicant: DARWIN PRECISIONS CORPORATION, Hsinchu County (TW)

(72) Inventors: Chung-Lin Lee, Hsinchu County (TW); YunPei Yang, Hsinchu County (TW)

(21) Appl. No.: 19/011,671

(22) Filed: Jan. 7, 2025

(30) Foreign Application Priority Data
Jan. 10, 2024 (TW) 113101082

(51) Int. Cl. A61M 37/00

(52) U.S. Cl. CPC A61M 37/0015; A61M 2037/0023; A61M 2037/0046; A61M 2205/0205; A61M 2205/0244

(57) ABSTRACT

An instant membrane includes a base layer and a microneedle layer. The base layer has a surface. The microneedle layer is disposed on the surface of the base layer and has a plurality of microneedles. The materials of the base layer and the microneedle layer include: 9 wt %-50 wt % polyvinylpyrrolidone K30, 40 wt %-90 wt % polyvinylpyrrolidone K90 and 0.09 wt %-9 wt % mini-hyaluronic acid.

### FIELD OF THE INVENTION

The invention provides a membrane, and in particular an instant membrane that has microneedles and can be quickly dissolved.

### BACKGROUND OF THE INVENTION

A microneedle patch is a transdermal drug delivery system, which penetrates into the stratum corneum of the skin through microneedles and delivers macromolecular drugs to the human body without stimulating pain nerves. Because the microneedle patch does not have to be metabolized by the digestive tract and liver, it can avoid the loss of efficacy caused by the first pass effect of the liver and can also deliver the drugs to the human body quickly.

The microneedles on the common microneedle patches on the market today are categorized into hollow microneedles, coated microneedles and dissolving microneedles. Compared with the hollow microneedles and the coated microneedles, the dissolving microneedles do not have the risk of drug accumulation that reduces the drug delivery efficiency, or microneedle breakage in the skin. However, the microneedle patch with dissolving microneedles needs to undergo mold filling, drying, demolding, etc. during the preparation process. During drying, the membrane surface is prone to curling or unflatness, so that the microneedle patch cannot be smoothly demolded and has poor releasability, thereby causing breakage of the membrane, skewing or breakage of the microneedles, and loss of the drug delivery function. Therefore, it is worthwhile to improve the yield of microneedle patches and increase the drug delivery rate.

### SUMMARY OF THE INVENTION

The invention provides an instant membrane, which has a flat and non-curling membrane surface during the preparation process, is easy to demold and has a high dissolution rate.

A instant membrane provided by the invention includes a base layer and a microneedle layer. The base layer has a surface, and the microneedle layer is disposed on the surface and has a plurality of microneedles. Materials of the base layer and the microneedle layer include: 9 wt %-50 wt % polyvinylpyrrolidone K30, 40 wt %-90 wt % polyvinylpyrrolidone K90 and 0.09 wt %-9 wt % mini-hyaluronic acid, and the mini-hyaluronic acid has an average molecular weight of 3000 to 10,000.

In an embodiment of the invention, the materials of the base layer and the microneedle layer further include 0.1 wt %-10 wt % first functional ingredient; and the first functional ingredient is selected from at least one of tetrapeptide, pentapeptide, hexapeptide, mandelic acid, arbutin, salicylic acid, tranexamic acid, vitamin B, vitamin C, ceramide, glucosides and derivatives thereof, or a composition thereof.

In an embodiment of the invention, the materials of the base layer and the microneedle layer further include 0.1 wt %-10 wt % second functional ingredient; and the second functional ingredient is selected from at least one of a biological agent, an analgesic, an antibiotic and an anti-inflammatory drug.

In an embodiment of the invention, the height of the microneedles is between 100 microns and 1500 microns.

In an embodiment of the invention, the microneedles have a shape of a pyramid or a cone.

The instant membrane of the invention uses the polyvinylpyrrolidone K30, the polyvinylpyrrolidone K90 and the mini-hyaluronic acid, and does not contain macromolecular hyaluronic acid at all. According to the proportions within specific ranges, the instant membrane has a flat and non-curling membrane surface during the preparation process, is easy to demold and has a high dissolution rate.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic side view of an instant membrane according to an embodiment of the invention;

FIG. 2 is a schematic three-dimensional view of an instant membrane according to an embodiment of the invention; and

FIG. 3 is a schematic three-dimensional view of an instant membrane according to another embodiment of the invention.

### DETAILED DESCRIPTION OF PREFERRED EMBODIMENTS

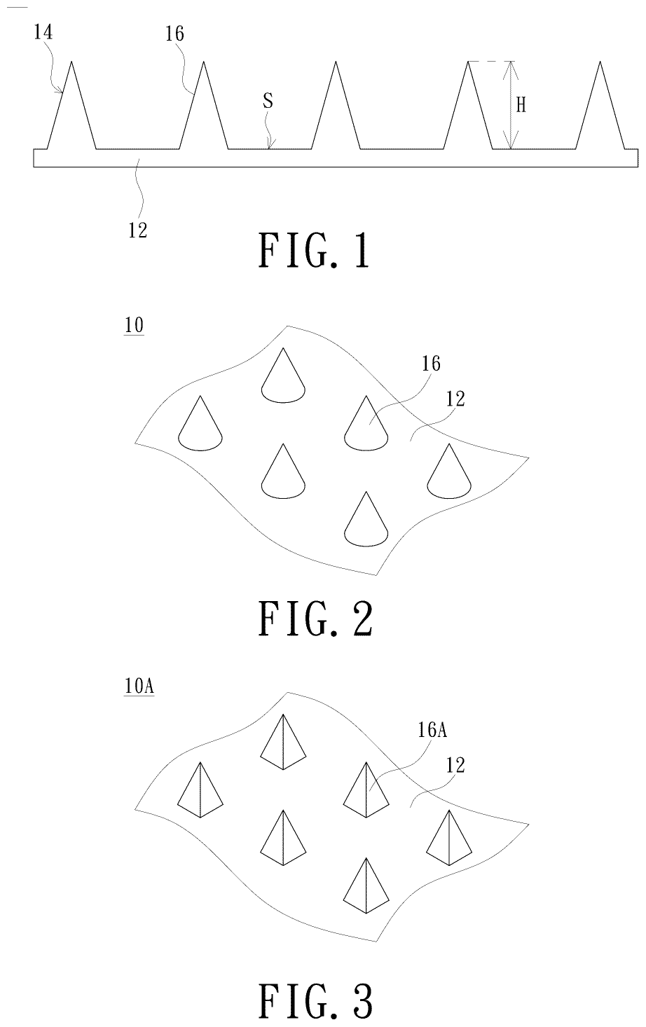

FIG. 1 is a schematic side view of an instant membrane according to an embodiment of the invention. As shown in FIG. 1, the instant membrane 10 includes a base layer 12 and a microneedle layer 14. The base layer has a surface S. The microneedle layer 14 is disposed on the surface S of the base layer 12. The microneedle layer 14 has a plurality of microneedles 16 spaced apart. In an embodiment, the microneedles 16 have a height H of 100 microns to 1500 microns. In an embodiment not shown, the instant membrane 10 further includes a peelable isolation layer, disposed on the other side of the base layer 12 away from the microneedle layer 14 to support or protect the instant membrane 10.

In an embodiment of the invention, the shape of the microneedles 16 of the instant membrane may be formed by using a mold. The instant membrane is prepared by the following steps: the materials of the base layer 12 and the microneedle layer 14 are dissolved in water to prepare an aqueous solution, and the aqueous solution is uniformly mixed and then poured into a mold; next, bubbles are removed, and 99% of water is removed by drying; and then, demolding is carried out for subsequent processing. The materials of the base layer 12 and the microneedle layer 14 include: 9 wt %-50 wt % polyvinylpyrrolidone K30, 40 wt %-90 wt % polyvinylpyrrolidone K90 and 0.09 wt %-9 wt % mini-hyaluronic acid. The polyvinylpyrrolidone K30 has an average molecular weight of 30,000 to 60,000, preferably 40,000. The polyvinylpyrrolidone K90 has an average molecular weight of 1,000,000 to 1,300,000, preferably 1,300,000. The mini-hyaluronic acid has an average molecular weight of 3000 to 10,000, preferably 5,500.

Table 1 shows test results of the materials of the base layer 12 and the microneedle layer 14 mixed with different solid weight percentages, and also shows the test results of the existing technologies using macromolecular hyaluronic acid as comparisons.

TABLE 1

| | | | Macro- | | | | | |
|---|---|---|---|---|---|---|---|---|
| | Polyvinyl- | Polyvinyl- | molecular | Mini- | | | 2-hour | |
| | pyrrolidone | pyrrolidone | hyaluronic | hyaluronic | Membrane | | disso- | |
| Sample | K90 (solid | K30 (solid | acid (solid | acid (solid | surface | Releas- | lution | |
| No. | wt %) | wt %) | wt %) | wt %) | flatness | ability | rate | Result |
| 1 | 75% | 0.0% | 25% | 0.0% | not curl | not break | 36% | Unacceptable |
| 2 | 85% | 0.0% | 15% | 0.0% | not curl | not break | 44% | Unacceptable |
| 3 | 90% | 0.0% | 10% | 0.0% | not curl | not break | 46% | Unacceptable |
| 4 | 0.0% | 75% | 25% | 0.0% | Curls | not break | 62% | Unacceptable |
| 5 | 0.0% | 85% | 15% | 0.0% | Curls | not break | 71% | Unacceptable |
| 6 | 0.0% | 90% | 10% | 0.0% | not curl | Breaks | 80% | Unacceptable |
| 7 | 89.9% | 9.9% | 0.0% | 0.09% | not curl | not break | 76% | Acceptable |
| 8 | 79.9% | 19.9% | 0.0% | 0.09% | not curl | not break | 82% | Acceptable |
| 9 | 49.9% | 49.9% | 0.0% | 0.09% | not curl | not break | 89% | Acceptable |
| 10 | 45.4% | 45.4% | 0.0% | 9.0% | not curl | not break | 60% | Acceptable |
| 11 | 0.0% | 88.9% | 0.0% | 11.1% | not curl | Breaks | 87% | Unacceptable |
| 12 | 81.8% | 9.0% | 0.0% | 9% | not curl | not break | 51% | Unacceptable |
| 13 | 72.0% | 18.0% | 0.0% | 9% | not curl | not break | 54% | Unacceptable |

Referring to Table 1, Sample 1 to Sample 3 are compositions of polyvinylpyrrolidone K90 and macromolecular hyaluronic acid in different proportions. During the drying and demolding process, their membrane surfaces do not curl (membrane surface flatness in Table 1), and these membranes do not break (releasability in Table 1), but their 2-hour dissolution rates are unsatisfactory. Sample 4 to Sample 6 are compositions of polyvinylpyrrolidone K30 and macromolecular hyaluronic acid in different proportions. Although their 2-hour dissolution rates are higher than those of Sample 1 to Sample 3, these membranes have poor membrane surface flatness (i.e., curl) or poor releasability (i.e., break). Sample 7 to Sample 10 are compositions of polyvinylpyrrolidone K90, polyvinylpyrrolidone K30 and mini-hyaluronic acid in different proportions. Their membrane surfaces do not curl (with reference to membrane surface flatness in Table 1), and these membranes do not break (with reference to releasability in Table 1); and moreover, their 2-hour dissolution rates are more than 60%, and particularly the 2-hour dissolution rate of Sample 9 is up to 89%. Sample 11 is a composition of polyvinylpyrrolidone K30 and mini-hyaluronic acid. Although its membrane surface does not curl and its 2-hour dissolution rate is up to 87%, this membrane has poor releasability. Sample 12 to Sample 13 are compositions of polyvinylpyrrolidone K90, polyvinylpyrrolidone K30 and mini-hyaluronic acid in different proportions, in which the proportions of the mini-hyaluronic acid are higher than those in Sample 7 and Sample 8, but their 2-hour dissolution rates are unsatisfactory.

The test results of the samples show that Sample 7 to Sample 10 each have good membrane surface flatness, releasability and 2-hour dissolution rate, and are suitable as the materials of the base layer 12 and the microneedle layer 14 in the instant membrane 10.

FIG. 2 is a schematic three-dimensional view of an instant membrane according to an embodiment of the invention. FIG. 3 is a schematic three-dimensional view of an instant membrane according to another embodiment of the invention. According to different structures of the mold used in the preparation steps, the microneedles have different shapes. As shown in FIG. 2, the microneedles 16 of the instant membrane 10 may have a shape of a cone. Alternatively, as shown in FIG. 3, the microneedles 16A of the instant membrane 10A may have a shape of a pyramid. The pyramid may be a triangular pyramid, a quadrangular pyramid, a hexagonal pyramid, etc., but it is not limited thereto.

In different embodiments of the instant membrane 10/10A of the invention, the materials of the base layer 12 and the microneedle layer 14 further include 0.1 wt %-10 wt % first functional ingredient; and a total content of the polyvinylpyrrolidone K30, the polyvinylpyrrolidone K90, the mini-hyaluronic acid, and the first functional ingredient does not exceed 100%. The first functional ingredient is selected from at least one of tetrapeptide, pentapeptide, hexapeptide, mandelic acid, arbutin, salicylic acid, tranexamic acid, vitamin B, vitamin C, ceramide, glucosides and derivatives thereof, or a composition thereof. The instant membrane 10/10A with the first functional ingredient can be effectively applied to the preparation of medicines for reducing melanin deposition, smoothing wrinkles, relieving skin inflammation, repairing wounds, etc.

In different embodiments of the instant membrane 10/10A of the invention, the materials of the base layer 12 and the microneedle layer 14 further include 0.1 wt %-10 wt % second functional ingredient; and a total content of the polyvinylpyrrolidone K30, the polyvinylpyrrolidone K90, the mini-hyaluronic acid, and the second functional ingredient does not exceed 100%. The second functional ingredient is selected from at least one of a biological agent, an analgesic, an antibiotic and an anti-inflammatory drug. The biological agent may be selected from at least one of a protein, a polypeptide, an amino acid or a nucleotide, and a cell extract. The anti-inflammatory drug may be selected from allantoin. The instant membrane 10/10A with the second functional ingredient can be effectively applied to the preparation of medicines for preventing or treating diseases and relieving symptoms such as pain or skin inflammation.

Table 2 shows solid weight percentages of the first functional ingredient and the second functional ingredient included in the instant membrane 10/10A of the invention.

TABLE 2

| | Content |
|---|---|
| Raw material | (solid wt %) |
| Polyvinylpyrrolidone K90 | 47.90% |
| Polyvinylpyrrolidone K30 | 41.90% |
| Mini-hyaluronic acid | 0.10% |
| First functional ingredient | 10.00% |
| Second functional ingredient | 0.10% |
| Total content | 100% |

The instant membrane uses the polyvinylpyrrolidone K30, the polyvinylpyrrolidone K90 and the mini-hyaluronic acid, and does not contain macromolecular hyaluronic acid at all. Moreover, the instant membrane is a mixture of ingredients in special proportions. Therefore, the instant membrane has a flat and non-curling membrane surface during the preparation process, is easy to demold and has a high dissolution rate. In addition, the microneedles have a height of 100 microns to 1500 microns, so that the special ingredient, such as the first functional ingredient or the second functional ingredient, can be delivered to the human body accurately, quickly and painlessly for a single site, thereby ensuring that the specific ingredient can maintain its medicinal properties.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An instant membrane, comprising a base layer and a microneedle layer, the base layer having a surface, and the microneedle layer being disposed on the surface and having a plurality of microneedles; wherein materials of the base layer and the microneedle layer comprise: 9 wt %-50 wt % polyvinylpyrrolidone K30, 40 wt %-90 wt % polyvinylpyrrolidone K90 and 0.09 wt %-9 wt % mini-hyaluronic acid, and the mini-hyaluronic acid has an average molecular weight of 3000 to 10,000.

2. The instant membrane according to claim 1, wherein the materials of the base layer and the microneedle layer further comprise 0.1 wt %-10 wt % first functional ingredient, and a total content of the polyvinylpyrrolidone K30, the polyvinylpyrrolidone K90, the mini-hyaluronic acid, and the first functional ingredient does not exceed 100%; and the first functional ingredient is selected from at least one of tetrapeptide, pentapeptide, hexapeptide, mandelic acid, arbutin, salicylic acid, tranexamic acid, vitamin B, vitamin C, ceramide, glucosides and derivatives thereof, or a composition thereof.

3. The instant membrane according to claim 1, wherein the materials of the base layer and the microneedle layer further comprise 0.1 wt %-10 wt % second functional ingredient, and a total content of the polyvinylpyrrolidone K30, the polyvinylpyrrolidone K90, the mini-hyaluronic acid, and the second functional ingredient does not exceed 100%; and the second functional ingredient is selected from at least one of a biological agent, an analgesic, an antibiotic and an anti-inflammatory drug.

4. The instant membrane according to claim 1, wherein the height of the microneedles is between 100 microns and 1500 microns.

5. The instant membrane according to claim 4, wherein the microneedles have a shape of a pyramid or a cone.
